(19) United States
(12) Patent Application Publication
(10) Pub. No.: US 2021/0052147 A1
(43) Pub. Date: Feb. 25, 2021

(54) EYEPIECE DEVICE FOR A SURGICAL INSTRUMENT

(71) Applicant: OLYMPUS WINTER & IBE GMBH, Hamburg (DE)

(72) Inventor: Gregor KIEDROWSKI, Hamburg (DE)

(73) Assignee: OLYMPUS WINTER & IBE GMBH, Hamburg (DE)

(21) Appl. No.: 16/964,634

(22) PCT Filed: Feb. 1, 2019
(86) PCT No.: PCT/EP2019/052482

§ 371 Date: Jul. 24, 2020

(30) Foreign Application Priority Data
Feb. 2, 2018 (DE) 10 2018 102 385.5

(51) Int. Cl. A61B 1/00; G02B 23/24; G02B 23/26; G02B 7/00

(52) U.S. Cl. CPC A61B 1/00195; A61B 1/00186; G02B 7/006; G02B 23/2453; G02B 23/26; A61B 1/00167

(57) ABSTRACT

An eyepiece device for a surgical instrument with an image guide channel part through which an image guide fibre bundle passes, the eyepiece device including: an optical filter disposed at a proximal end of the image guide fibre bundle; a sliding body having the optical filter element disposed therein; and a connector disposed at a proximal end of the image guide channel part; wherein the sliding body is disposed longitudinally displaceable within the connector.

### CROSS-REFERENCE TO RELATED APPLICATION

The present application is based upon and claims the benefit of priority from PCT/EP2019/052482 filed on Feb. 1, 2019, which claims benefit to DE 10 2018 102 385.5 filed on Feb. 2, 2018, the entire contents of each of which are incorporated herein by reference.

### BACKGROUND

### Field

The present disclosure relates to an eyepiece device and more particularly to an eyepiece device for use in surgical instruments as well as surgical instruments, which include the eyepiece device.

### Prior Art

For minimally invasive interventions on the human or animal body, endoscopic instruments are usually used, which allow an optical system to observe the operation or examination field. For this purpose, the instruments usually have an optical system spanning the tubular shaft, in which a lens is disposed in the distal end region of the instrument and an eyepiece device is disposed in the proximal end region of the instrument. Between the lens and the eyepiece extends an image transmission device, for example, an image guide fibre bundle. Corresponding eyepiece devices are described in, for example, DE 10 2015 202 002 A1 and DE 2015 202 137 A1.

The eyepiece device can be used directly to observe the site of intervention with the naked eye. Alternatively, however, often a camera head is also connected to the eyepiece so that the intervention can be observed on a monitor and the visual data can be supplied to a further digital processing (see EP 0 501 088 A1). For the transmission of visual data from the operation or examination field, image guide fibre bundles are preferred in particular to lens systems when in surgical instruments with a thin shaft part an image transmission unit with a particularly small diameter is required.

In the image transmission by image guide fibre bundles, optical interferences, e.g. moire effects, occur. Such moire effects occur, for example, due to interferences generated within the fibre bundle or in the connection between the fibre bundle and an electronic image pickup element (hereinafter referred to as “CCD”). To reduce the moire effects, optical filters can be used (anti-moire filters). Anti-moire filters can, for example, consist of 3 or more glass layers and are described, for example, in U.S. Pat. No. 6,025,873. The connection between the filter and the image guide fibre bundle can be produced, for example, on the end face by an inflexible optical adhesive. In addition, the filter is adhered by means of a flexible adhesive within the instrument. The flexible adhesive is used to fill in the uneven gap. Due to the flexible adhesive, the support necessary due to the relatively high weight of the filter and the low connecting surface can be ensured. The support is made flexible, since the different thermal expansion properties of the metal, glass and plastic components could otherwise lead to the destruction of the connection between the fibre bundle and the filter. As flexible adhesives, silicone adhesives, for example, are currently used.

At the same time, however, inflexible epoxy resin adhesives are also used within the surgical instruments in the immediate vicinity of this bonding site, e.g. to ensure a sealing bond to the instrument exterior. The simultaneous use of silicone and epoxy resin adhesives is associated with a high cost, since both types of adhesives must be applied strictly separated from each other and cured so as not to adversely affect the adhesive strength of the other adhesive. Thus, contamination with a wafer-thin layer of silicone adhesive under an epoxy adhesive can cause it to fail to bond firmly to the substrate. Vice versa, the epoxy adhesive can also affect the cross-linking of the silicone adhesive, depending on the curing agent component used. Platinum catalysts, for example, which can be used in silicone adhesives, react sensitively to amine-containing epoxy adhesive curing agents. Since even in the curing of epoxy adhesives escaping gases may be sufficient to negatively affect the cross-linking of an adjacent silicone adhesive, the two types of adhesive usually have to work in separate spaces and separate ventilation systems.

### SUMMARY

An object would therefore be to dispense with the use of a flexible silicone adhesive in the immediate vicinity of the necessary epoxy bondings to reduce the production effort and completely eliminate possible mutual contamination of the adhesives.

Such object can be achieved by an eyepiece device, for a surgical instrument, such as an endoscope, with an image guide channel part through which an image guide fibre bundle passes, an optical filter element at the proximal end of the image guide fibre bundle and a connecting element, which is disposed at the proximal end of the image guide channel part, wherein the optical filter element is disposed in a sliding element and the sliding element is disposed longitudinally displaceable within the connecting element.

By attaching the optical filter element in a longitudinally displaceable sliding element, the different thermal expansion properties of processed glass, metal and plastic materials can be compensated by axial movements of the sliding element. The risk of a separation of the optical filter element from the image guide fibre bundle by the different expansion of materials used is thereby minimized The use of a flexible adhesive can be dispensed with, so that in the course of the production process no negative interactions between flexible (epoxy) adhesives and inflexible (silicone) adhesives can occur.

Further, the relatively heavy optical filter can be supported radially, since it can be securely received in the sliding element and the sliding element in turn can pass precisely within a connecting element. The mating connection between the sliding element and the connecting element can permit only axial movements of the sliding element.

Eyepiece devices and surgical instruments comprising such eyepiece devices can be classified into two different types, such as a lens relay type that uses lenses in an optical image transmission system, and in a fibre relay type that uses a fibre bundle for image transfer. The eyepiece device disclosed in the embodiments is an eyepiece device of the fibre relay type.

In eyepiece devices of the fibre relay type, when the image guide fibre bundle is combined for the image transmission from the intervention site with a camera head to record the image data, unwanted moire effects regularly occur. The eyepiece device is the eye or the camera head-side part of the optical system of a surgical instrument, which adjoins the image transmission device proximally. In this case, the eyepiece device can comprise the proximal end region of the image transmission device, e.g. the image guide fibre bundle.

In addition, the eyepiece device can comprise an optical filter element. The filter element can be configured for the optical suppression of moire effects, such as moire effects which have been generated by the use of the image guide fibre bundle for the image transmission from the intervention site and/or by the combination of image guide fibre bundles with a camera head. Thus, the filter element can be an anti-moire filter or an optical low-pass filter. However, other optical filter elements can be used in the eyepiece device. In general, the filter elements can improve the image quality and/or simplify the evaluation of the transmitted image information. This does not exclude that the use of the filter elements may reduce the sharpness of the image.

Optical filter elements, such as low-pass filters or anti-moire filters, can have different structures. In general, however, the filter element can have three or more glass layers with different refractive properties. Corresponding filter elements, such as anti-moire or low-pass filters, are known to those having ordinary skill in the art.

The optical filter element can be disposed at the proximal end of the image guide fibre bundle. The optical filter element can be directly adjacent to the image guide fibre bundle or separated from the image guide fibre bundle only by one adhesive layer. For bonding the image guide fibre bundles and filter element, adhesives suitable for optics can be used. These can be inflexible adhesives, such as epoxy resin adhesives. The adhesive can be applied to the front side (the proximal end) of the image guide fibre bundle. An inflexible (solid) connection thus results between the image guide fibre bundle and the filter element, so that fibre bundles and filter element are not movable relative to each other.

The eyepiece device can have an image guide channel part through which the image guide fibre bundle passes. The image guide channel part may be tubular or hollow cylindrical. This means that the image guide channel part can have an elongated cavity or channel for receiving the image guide fibre bundle. The image guide channel part can pass through the entire shaft part in a surgical instrument.

The image guide channel part, such as the exterior of the image guide channel part, can deviate from a strict hollow cylindrical shape. Thus, the image guide channel part can have, for example, in its proximal end region one or a plurality of fastening elements. The fastening elements can be configured so that the connecting element described elsewhere can be attached to them. For this purpose, the fastening element(s) can be formed, for example, as an external thread, as a groove or as a tongue (to form a screw, tongue-and-groove or bung connection).

The image guide fibre bundle disposed in the image guide channel part can be part of an image transmission system that transmits an image from the surgical site to the proximal end of the surgical instrument. Suitable image guide fibre bundles are known to those having ordinary skill in the art and include, for example, fibre optic image guides.

The optical filter element disposed at the proximal end of the image guide fibre bundle can be disposed in a sliding element. The sliding element can enclose the optical filter element along the entire circumference of the filter element, but not the end faces of the filter element. The sliding element can also have a hollow cylindrical shape for this purpose, in the interior of which the optical filter element is disposed. This means that the sliding element can have a short cylindrical cavity or channel for receiving the optical filter element.

The sliding element can be disposed proximally to the image guide channel part in extension of its longitudinal axis. For connection and attachment of sliding element and image guide channel part to each other, the eyepiece can have a connecting element.

The connecting element can be disposed in the proximal end region of the image guide channel part and can be connected or connectable there to the image guide channel part. The connecting element can be configured as a union nut.

The connecting element can have in its distal end region one or a plurality of fastening elements which can be at least partially complementary in shape to the fastening elements on the image guide channel part. With the help of the respective fastening elements, the connecting element and image guide channel part can be connectable to each other. The fastening element(s) can, for example, be formed as an internal thread on the connecting element. In this case, the connecting element can be configured as a union nut (with internal thread), whose internal thread in the mounted state meshes with an external thread disposed on the image guide channel part (and thus producing a screw connection). Alternatively, the fastening element(s) can be formed on the connecting part as a tongue or groove for producing a tongue-and-groove or bung connection with a groove or tongue on the image guide channel part.

The sliding element can be disposed longitudinally displaceable within the connecting element. This axial displaceability of the sliding element can ensure that different thermal expansions of the processed components are compensated by a longitudinal displacement of the sliding element and not by a lost connection between the filter element and the fibre bundle. The axial displaceability basically can allow both an axial movement of the sliding element to distally and proximally. The sliding movement can, for example, be limited by abutment surfaces described elsewhere.

While axial movements of the sliding element and the filter element can be provided, lateral movements can be prevented. Therefore, the sliding element can be supported radially by a fitting connection with the connecting element. For this purpose, the sliding element can comprise a distal section, which forms a fitting connection with a proximal section of the connecting element. The outside of the sliding element can adjoin the inside of the connecting element substantially directly in this section. The fitting connection can be configured such that it allows an axial displacement of the sliding element, but ensures sufficient support radially. For this purpose, the fitting connection can be a clearance or a transitional fitting connection, wherein the clearance connection has no significant play (e.g., DIN 7154, clearance H7/g6).

A clearance H7/g6 according to DIN 7154 can be provided. The different expansions caused by different thermal expansion properties can be sufficient to cause an axial displacement of the sliding element.

The connecting element and/or the sliding element can be made of a plastic or metal. Suitable plastics and metals are known and include, for example, PEEK (polyether ether ketone) or brass. The formation of both elements from plastic can be used.

The filter element can also be longitudinally displaceable together with the sliding element. At the same time, the filter element can not be movable or displaceable relative to the sliding element. For this purpose, the optical filter element can be connected by means of an adhesive layer with the sliding element. In other words, the filter element and the sliding element can be bonded together. The filter element can be bonded along its circumference, such as completely along its circumference, at its outer edge to the inside of the sliding element.

Since the use of flexible adhesives, such as the use of silicone adhesives, can be avoided, the adhesive used for the adhesive layer can be an inflexible adhesive. The inflexible adhesives can be epoxy resin adhesives.

The axial displaceability of the sliding element can be limited by abutment surfaces. For this purpose, the image guide channel part and/or the connecting element can comprise an abutment (abutment surface) for the sliding element, which can limit a movement of the sliding element in the axial direction. In one embodiment, the image guide channel part can include an abutment for the sliding element that limits movement of the sliding element in the distal direction. The abutment can be formed by a section of the proximal end face of the image guide channel part. Alternatively or additionally, the connecting element can comprise an abutment for the sliding element, which can limit a movement of the sliding element in the distal direction. In such an embodiment, the sliding element can have a widened side edge in its proximal end region, which can have a contact surface for the abutment of the connecting element. The abutment of the connecting element can, for example, be formed by a section of the proximal end face of the connecting element or by the entire end face. The connecting element can comprise a (further) abutment for the sliding element, which can limit a movement of the sliding element in the proximal direction. In this case, the sliding element can be arranged completely within the connecting element.

Also provided is a surgical instrument, such as an endoscope, wherein the surgical instrument comprises an eyepiece device. The surgical instrument can be an endoscope, for example an endoscope selected from the group consisting of laparoscopes, gastroscopes, colonoscopes, bronchoscopes and the like. The surgical instrument can be an instrument (e.g. endoscope) of the fibre relay type (fibrescope), such as a fibre optic endoscope.

### BRIEF DESCRIPTION OF THE DRAWINGS

In the drawings, embodiments are shown schematically. In the following:

FIG. 1 illustrates a schematic and simplified side view of a surgical instrument;

FIG. 2 illustrates a schematic and simplified sectional view of an eyepiece device; and

FIG. 3A illustrates the representation of FIG. 2 in reduced form and a corresponding, simplified sectional view of the eyepiece device, which allows a view from the proximal direction in FIG. 3B.

### DETAILED DESCRIPTION

Further advantages, characteristics and features will become apparent in the following detailed description of embodiments with reference to the accompanying drawings. However, the invention is not limited to these exemplary embodiments.

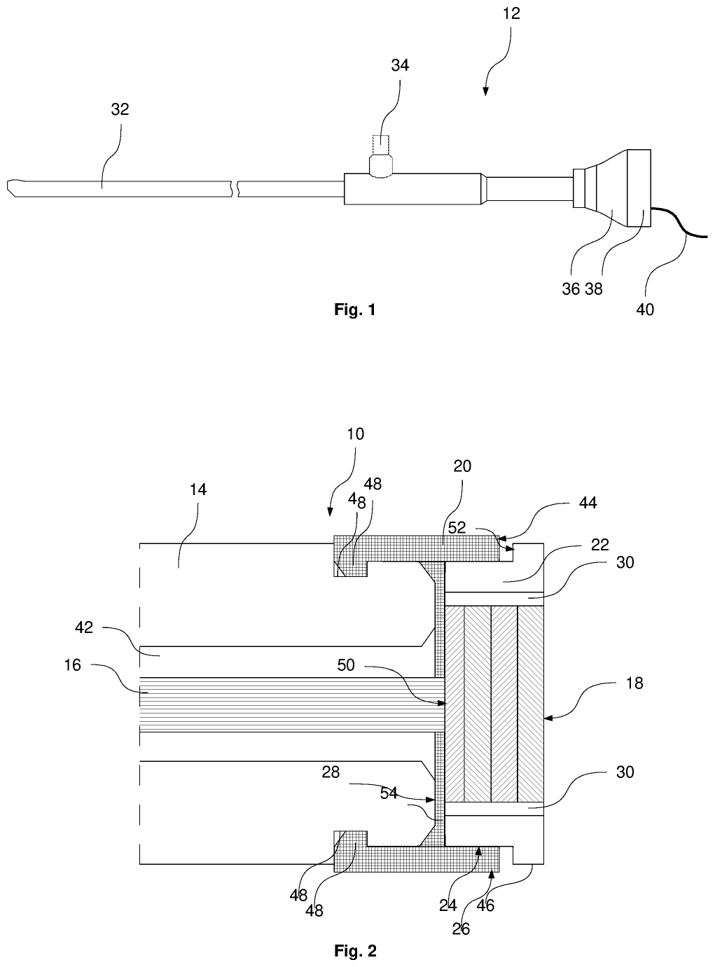

FIG. 1 shows a schematic and simplified side view of a surgical instrument 12, which is configured as a rigid endoscope. It is understood that the eyepiece device 10, not shown in FIG. 1, is equally suitable for flexible endoscopes. The surgical instrument 12 has a tubular shaft part 32 and a light guide input port 34. By the latter, a light guide for illuminating the site of intervention can be connected to the endoscope. A corresponding light guide bundle runs in a manner not shown here through the endoscope. In addition to this illumination device and optional working channels, the endoscope has an optical system for monitoring the site of intervention. The illustrated instrument is of a fibre relay endoscope type and accordingly has an image guide fibre bundle, not shown here, for image transmission between the site of intervention and the camera head 38. The optical system further comprises an eyepiece funnel 36 and a connector 40 to an image processor.

FIG. 2 shows a schematic and simplified sectional view of an eyepiece device 10, which is suitable for use in the endoscope shown in FIG. 1. The eyepiece device 10 has an image guide channel part 14, a connecting element (connector) 20 and a sliding element (sliding body) 22.

The image guide channel part 14 is a substantially hollow cylinder. In the interior of the image guide channel part 14, a channel 42 is formed, through which the image guide fibre bundle 16 passes. The channel 42 extends into the distal end region of the surgical instrument 12, not illustrated here. The image guide fibre bundle 16 can be a fibre optic bundle. The image guide fibre bundle 16 may be protected within the channel 42 in a manner not shown by adjacent elements. For example, the image guide fibre bundle 16 may be enclosed by a channel or hose element.

In its proximal end region, the image guide channel part 14 has a fastening element 48, which in this case is configured as a circumferential groove. A section of the proximal end face of the image guide channel part 14 serves as an abutment surface, which limits the movement of the sliding element 22 in the distal direction.

At its proximal end face 50, the image guide fibre bundle 16 is connected to the filter element (optical filter) 18 by means of an optical adhesive (such as an epoxy resin adhesive). The filter element 18 is an anti-moire filter consisting of four glass layers, three of which are effective in reducing moire effects. The filter element 18 is provided on its outer circumference with an adhesive layer 30, by means of which it is connected to the cylindrical inner surface of the sliding element 22. The adhesive layer 30 can comprise a cured epoxy resin adhesive. The adhesive is an inflexible adhesive.

The sliding element 22 has in its proximal end region a widened side edge 46 (holding shoulder) which comprises a contact surface pointing distally. The contact surface 52, together with the abutment 44 of the connecting element 20, limits the distal movement of the sliding element 22.

The connecting element 20 serves to connect the image guide channel part 14 and the sliding element 22. The connection between the connecting element 20 and the image guide channel part 14 is fixed, so that the parts do not move relative to one another in the mounted state. For this purpose, the connecting element 20 has one or a plurality of (helical) protuberances extending in the interior of the connecting element 20, by means of which a screw connection between the connecting element 20 and the image guide channel part 14 can be produced. Alternatively, it is also conceivable to use a bung connection. To facilitate assembly, the connecting element 20 may be made of a plastic having a flexibility, for example, to push the connecting element 20 from the proximal direction for producing a bung connection to the image guide channel part 14. In contrast, the connection between the connecting element 20 and the sliding element 22 is configured such that a radial displacement of the sliding element 22 within the connecting element 20 is prevented, while an axial movement of the sliding element 22 in the interior of the connecting element 20 is made possible.

For this purpose, the connecting element 20 has an at least partially hollow cylindrical configuration, wherein the sliding element 22 is longitudinally displaceable in the interior of the connecting element 20. The longitudinal displacement of the sliding element 22 is limited by the connection between the image guide fibre bundle 16 and the sliding element 22. Since the image guide fibre bundle 16 can be fixed distally, the maximum axial movement distance of the sliding element 22 is limited to the extent of thermal expansion of the fibre bundle 16 in relation to the thermal expansion of the remaining components used. Between the proximal end of the image guide channel part 14 and the distal end of the optical filter element 18, the adhesive layer 30 and the sliding element 22, a gap 54 is formed. The size of the gap 54 changes with the longitudinal displacement of the sliding element 22. The gap 54 may, for example, be an air gap.

The sliding element 22 is disposed (radially) in a fitting connection in the connecting element 20. This ensures that the sliding element 22 is longitudinally but not radially displaceable. The outer circumference of the sliding element 22 adjoins the inner surface of the connecting element 20 during a longitudinal displacement.

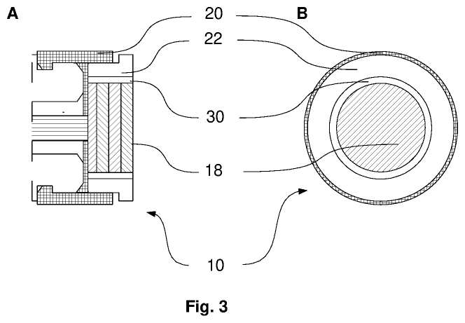

FIG. 3A shows the sectional view of FIG. 2 in reduced form and beyond a corresponding rotated sectional view of the eyepiece device 10, which allows a view from the proximal direction in FIG. 3B. It can be seen that in the illustrated embodiment, the optical filter element 18, as well as the other elements, have a round or cylindrical cross-sectional shape.

While there has been shown and described what is considered to be preferred embodiments of the invention, it will, of course, be understood that various modifications and changes in form or detail could readily be made without departing from the spirit of the invention. It is therefore intended that the invention be not limited to the exact forms described and illustrated, but should be constructed to cover all modifications that may fall within the scope of the appended claims.

### LIST OF REFERENCE NUMERALS

10 eyepiece device

12 surgical instrument

14 image guide channel part

16 image guide fibre bundle

18 optical filter element

20 connecting element

22 sliding element

24 distal section of sliding element

26 proximal section of connecting element

28 abutment

30 adhesive layer

32 shaft part

34 light guide input port

36 eyepiece funnel

38 camera head

40 connector

42 channel

44 abutment

46 widened side edge

48 fastening element

50 end face

52 contact surface

54 gap

What is claimed is:

1. An eyepiece device for a surgical instrument with an image guide channel part through which an image guide fibre bundle passes, the eyepiece device comprising:
an optical filter disposed at a proximal end of the image guide fibre bundle;
a sliding body having the optical filter element disposed therein; and
a connector disposed at a proximal end of the image guide channel part;
wherein the sliding body is disposed longitudinally displaceable within the connector.

2. The eyepiece device according to claim 1, wherein the sliding body comprises a distal section, which forms a fitting connection with a proximal section of the connector.

3. The eyepiece device according to claim 1, further comprising an adhesive for connecting the optical filter to the sliding body.

4. The eyepiece device according to claim 3, wherein the optical filter is bonded along at least a portion of a circumference of the optical filter to the sliding body by the adhesive.

5. The eyepiece device according to claim 3, wherein the adhesive is an inflexible adhesive.

6. The eyepiece device according to claim 1, wherein the connector include a thread for mating engagement with a thread on the proximal end of the image guide channel part.

7. The eyepiece device according to claim 1, wherein one or more of the connector and the sliding body is formed of plastic.

8. The eyepiece device according to claim 1, wherein the connector comprises an abutment for for limiting a movement of the sliding body in the axial direction.

9. The eyepiece device according to claim 8, wherein the sliding element comprises a widened side edge having a contact surface for engagement with the abutment of the connector.

10. A surgical instrument comprising:
the image guide channel part; and
the eyepiece device according to claim 1 disposed on the proximal end of the image guide channel part.

11. The eyepiece device according to claim 4, wherein the optical filter is bonded completely along the circumference of the optical filter to the sliding body by the adhesive.

12. The eyepiece device according to claim 5, wherein the inflexible adhesive is an epoxy adhesive.

13. The eyepiece device according to claim 6, wherein the thread on the connector is an internal thread.
